(19) United States
(12) Patent Application Publication
(10) Pub. No.: US 2023/0398051 A1
(43) Pub. Date: Dec. 14, 2023

(54) Anti-Microbial Skin-Care Composition Comprising Beta-Glucan and Natural Oil

(71) Applicant: LANTMÄNNEN OATS AB, Kimstad (SE)

(72) Inventors: Torsten Hedbom, Borlänge (SE); Lars Franzén, Stockholm (SE)

(73) Assignee: LANTMÄNNEN OATS AB, Kimstad (SE)

(21) Appl. No.: 18/250,643

(22) PCT Filed: Nov. 18, 2021
(86) PCT No.: PCT/EP2021/082135

§ 371 Date: Apr. 26, 2023

(30) Foreign Application Priority Data
Nov. 18, 2020 (SE) 2051346-1

(51) Int. Cl. A61K 8/73; A61K 8/36; A61K 8/22; A61K 8/92; A61K 8/19; A61K 8/67; A61Q 17/00; A61Q 19/00; A61K 8/9794

(52) U.S. Cl. CPC A61K 8/73; A61K 8/36; A61K 8/22; A61K 8/922; A61K 8/19; A61K 8/67; A61Q 17/005; A61K 8/737; A61Q 19/007; A61K 8/9794

(57) ABSTRACT

The present invention relates to a skin-care composition comprising beta-glucan and a natural oil, as well as either one or both of hydrogen peroxide and lactic acid. The skin-care composition is mainly based on natural ingredients. The composition has biocidal/anti-microbial activity and is very skin-friendly and non-irritative. The skin-care composition contains beta-glucan, which has an emulsifying effect, as well as a smoothening and moisturizing effects on the skin. A skin-care composition of the present invention does not negatively affect the barrier function of the skin.

### FIELD

The present invention relates to a skin-care composition comprising beta-glucan and a natural oil, as well as either one or both of hydrogen peroxide and lactic acid. The skin-care composition is mainly based on natural ingredients. The composition has biocidal/anti-microbial activity and is very skin-friendly and non-irritative. It can be used to remove unwanted microorganisms such as bacteria or viruses present on the skin of a subject to avoid or minimize the risk of infecting the subject with the microorganism. The skin-care composition contains beta-glucan, which has an emulsifying effect, as well as a smoothening and moisturizing effects on the skin. A skin-care composition of the present invention does not negatively affect the barrier function of the skin.

### BACKGROUND

The skin is the largest organ of the human body and hosts many different microorganisms. Normally such microorganisms are harmless to the human body, but in certain situations the skin also hosts pathogens. Pathogens on the skin are known to cause illness and may be easily transmitted from one person to another. In the pandemic situation with Covid-19, one of the important measures is to keep distance from each other and to use the traditional approach of skin cleaning and disinfection such as washing with soap or using ethanol-based compositions such as gels. Alcohol-based hand sanitizers typically contain some combination of isopropyl alcohol, ethanol or n-propanol with versions containing 60% to 95% ethanol being most effective. Care should be taken as they are flammable. Alcohol-based hand sanitizers work against a wide variety of microorganisms, but not spores. Compounds such as glycerol may be added to prevent drying of the skin. Some versions contain fragrances; however, these are discouraged due to the risk of allergic reactions. Non-alcohol-based versions typically contain benzalkonium chloride or triclosan but are less effective than the alcohol-based ones.

Some of the drawbacks with the traditional approach of skin cleaning and disinfection is the tendency of drying out the skin and the tendency of obtaining an irritated or red skin due to the frequent cleaning process. Alcohol may also strip the skin of the outer layer of oil, which may have negative effects on the barrier function of the skin. There is therefore a need for providing a skin-care composition that can remove or kill the unwanted pathogens from the skin without the drawbacks of the compositions traditionally employed.

### SUMMARY

The present invention provides a skin-friendly skin-care composition comprising beta-glucan a natural oil and one or both of hydrogen peroxide and lactic acid. A skin-care composition of the invention is advantageous in that it has no or minimal impact on the barrier function of the skin; thus, the barrier function of the skin remains intact even after repeated use of a skin-care composition of the present invention.

In one embodiment, a skin-care composition of the present invention comprises beta-glucan, a natural oil and hydrogen peroxide, in one embodiment, a skin-care composition of the present invention comprises beta-glucan, a natural oil and lactic acid and, in one embodiment, a skin-care composition of the present invention comprises beta-glucan, hydrogen peroxide, lactic acid and a natural oil, wherein the concentration of the natural oil is in a range of from 3 to 15% w/w based on the weight of the composition and wherein the concentration of the beta-glucan is from 0.3 to 20% w/w. Such a skin-care composition is in the form of an oil-in-water emulsion.

Typically, the concentration of lactic acid is from 0.1 to 5% w/w such as from 0.25% to 5% w/w or from 1% to 5% w/w and/or the concentration of hydrogen peroxide is from 0.5 to 15% w/w.

The content of beta-glucan in a skin-care composition of the invention contributes to the overall skin-friendly appearance of the composition when applied to the skin. Thus, beta-glucan is believed to have smoothening and softening properties and, moreover, it contributes to the texture of the composition (e.g. as a surfactant, an emulsifier or it contributes to the viscosity of the composition). Further, the composition contains a natural oil. The concentration of the natural oil is in a range of from 3 to 15% w/w based on the weight of the composition.

A skin-care composition according to the invention is formulated mainly with natural agents. Thus, in general at least 85% w/w of the ingredients are natural ingredients.

A composition of the present invention contains one or more antibacterial, antiviral and/or antifungal agents. Lactic acid is known to possess antimicrobial properties and skin-friendly properties and it is used in a composition of the invention. The same applies to hydrogen peroxide, which also has antimicrobial activity. Beta-glucan and the natural oil provide moistening and smoothening properties to the skin.

Beta-glucans are polysaccharides of D-glucose monomers linked by beta-glycosidic bonds. They occur naturally in some yeast. Fungi, plants and bacteria. Beta-glucans occur in various forms such as (1,3)-beta-glucan, (1,4)-beta-glucan, (1,6)-beta-glucan, (1,3:1,6)-beta-glucan and (1,3:1,4)-beta glucan. Beta-glucan are notably found in cereal grains such as in wheat, barley, rye and oat. Oats are a particularly good source of (1,3:1,4)-beta-glucan. As seen from the examples herein, a beta-glucan containing composition may be used such a PromOat® that contains water-soluble (1,3:1,4)-beta-glucans with a molecular weight in a range of from 0.8 to 2.5 MDa. Based on these examples, it is contemplated that beta-glucans suitable for use according to the invention are i) water-soluble beta-glucans, ii) (1,3:1,4)-beta-glucans, and/or iii) beta-glucans having a molecular weight in a range of from 0.1 to 3.0 MDa such as in a range of from 0.5 to about 2-8 MDa or in a range of from about 0.8 to about 2.5 MDa.

The concentration of beta-glucan in a skin-care composition of the invention is generally in a range of from 0.3% to 20% w/w based on the total weight of the skin-care composition.

In cosmetics, beta-glucans without significant contamination with other components should be used. Beta-glucans can be obtained by several processes, see e.g. WO 2016/124608, which describes a method for purifying a mixture containing beta-glucan.

Hydrogen peroxide works by producing hydroxyl free radicals that can attack membrane lipids, DNA and other essential cell components. Hydrogen peroxide is active against a wide range of microorganisms including bacterial, fungi, viruses and spores. A 0.5% hydrogen peroxide has demonstrated bactericidal and virucidal activity in 1 minute. In general concentrations up to 25% are considered suitable as chemical sterilisants.

Lactate may be a suitable salt of lactic acid, which may be D-lactic acid, L-lactic acid or D,L-lactic acid, or it may be in the form of lactic acid. The salt may be used in the form of an alkaline metal salt or an alkaline earth metal salt of lactic acid. Dependent of pH of the composition lactate may be present as lactate, lactic acid or a mixture thereof.

Lactic acid is a safe, biobased and biodegradable agent with proven broad range efficacy against bacteria and viruses. The concentration of lactic acid in a composition of the invention is a concentration where it has antimicrobial properties and still does not make any harm to the skin. In general, a concentration of 0.1% w/w or more is suitable such as 0.25% w/w or more or 0.5% w/w or more. In general, a concentration of 0.25% w/w or more is regarded as bactericidal. Normally, the concentration of lactic acid is in a range of from 0.1% to 5% w/w, such as from 0.25% to 5% w/w, from 0.5% to 5% w/w, from 0.5% to 3% w/w such as about 1.5% w/w or 2.0% w/w. However, if more than one antibacterial, antiviral or antifungal agent are present, the concentration of lactic acid may be lowered. In order to obtain a composition that is consumer-friendly, the concentration of lactic acid should not be too high. In general, the concentration of lactic acid should be at the most 5-10% w/w.

The content of a natural oil contributes to the overall appearance and sense of a skin-friendly composition. Moreover, it is present as the oil phase in as an oil-in-water emulsion is formed. In the composition of the invention, beta-glucan functions as an emulsifier linking and stabilizing the oil and water phases together. The concentration of the natural oil in a composition of the invention is from about 3 to 15% w/w and even if the content is relatively low, it is possible to obtain a stable emulsion in compositions where water is present due to the emulsifying and stabilizing effect of the beta-glucan.

Other ingredients in the skin-care composition are generally natural ingredients. Specifically, the vehicle used as basis for the skin-care composition is processed cereals, preferably processed oats, where parts of the hull of the oat have been removed. Beta-glucan is typically provided admixed with the powdered processed oat, or the mixture results from purification of oats. In the following such a mixture is denoted beta-glucan composition. The beta-glucan composition typically has a content of beta-glucan of from 32 to 36% w/w. The beta-glucan composition is preferably derived from cereals, notably from oats and has a protein content of from 2 to 5% w/w and a total carbohydrate content of from 75 to 90% w/w (i.e. including the content of beta-glucan).

When such a beta-glucan composition is used it is possible to obtain a skin-care composition having a content of natural ingredients in a concentration of 80% w/w or more such as 85% w/w or more, 90% w/w or more, 92% w/w or more, 93% w/w or more, 94% w/w or more or about 95% w/w. As seen from the examples herein a skin-care composition of the invention may have a content of 95% or more of natural ingredients; thus, negative effects from artificial ingredients like e.g. preservatives or other substances that may be allergenic or irritative to the skin can be avoided.

In one embodiment of the present invention, the skin-care composition comprises beta-glucan, lactic acid and a natural oil and no hydrogen peroxide, wherein the concentration of the natural oil is in a range of from 3 to 15% w/w based on the weight of the composition and wherein the concentration of the beta-glucan is from 0.3 to 20% w/w and wherein the concentration of lactic acid is from 0.1 to 5% w/w such as from 0.25% to 5% w/w or from 1% to 5% w/w.

A skin-care composition of the present invention may be used as a disinfectant composition to kill or deactivate unwanted microorganisms present on the skin, especially pathogenic microorganism. The composition is especially useful as a hand sanitizer for hand disinfection.

### Definitions

The term “microorganism” or “microbe” is a microscopic organism, which may exist in its single-celled form or a colony of cells. Microorganisms can be found almost anywhere on earth and include both prokaryotes and eukaryotes. Microorganisms may be Archaea, Bacteria, Eukaryotes, Protists, Fungi or Plants. Viruses are generally regarded as not living and therefore not considered as microorganisms.

A virus is a submicroscopic infectious agent that replicates only inside the living cells or an organism. Viruses infect all types of life forms, from animals and plants to microorganism including bacteria and archaes. When infected, a host cell is forced to rapidly produce thousands of identical copies of the original virus. Viruses are spread in many ways. One transmission pathway is through disease-bearing organisms or it can be spread e.g. by coughing and sneezing.

Antivirals are a class of antimicrobials and are described in the following.

The term “antimicrobial” is used in the present context as an agent that kills microorganisms or stop their growth. Antimicrobials are effective against e.g. bacteria, fungi and/or viruses.

Antimicrobial medicines can be grouped according to the microorganisms they act primarily against. For example, antibiotics are used against bacteria, antivirals are used against viruses, and antifungals are used against fungi. Agents that kill microbes are microbiocidal, while those that mere inhibit their growth are call biostatic; analogously the term bacteriocidal refers to agents than kill bacteria and the term bacteriostatic refers to agent that merely inhibit the growth of bacterial.

The terms virucidal and virustatic relate to viruses. Virucidal refers to agents that deactivate or destroy viruses, and virostatic relates to agents that inhibit cell entry and/or replication, usually by inhibiting binding to cell receptors or interfering with a biological process The use of antimicrobial medicines to treat infection is known as antimicrobial chemotherapy, while the use of antimicrobial medicines to prevent infection is known as antimicrobial prophylaxis. In its broadest meaning, a composition of the present invention is antimicrobial, i.e. it is an antibacterial composition such as a bacteriocide and/or bacteriostatic composition, and/or an antiviral composition such as a virucidal composition and/or a virustatic composition.

The term “disinfectant” as used herein are chemical agents designed to inactivate or destroy microorganisms on inert surfaces.

The term “hand sanitizer” as used herein is a liquid, gel or foam generally used to decrease infectious agents on the hands. In most healthcare setting alcohol-based hand sanitizers are preferable to hand washing with soap and water because it may be better tolerated and is more effective at reducing bacteria.

The term “pathogen” as used herein is anything that can produce a disease. A pathogen may also be referred to as an infectious agent. Pathogens include algae, bacteria, fungi, prions, viroids, viruses and other parasites.

The term “treatment” as used herein is intended to mean implementation of therapy with the intention of reducing the severity or frequency of symptoms. As used herein the term “treatment” refers both to therapeutic treatment and prophylactic or preventive measures.

The term “prevention” as used herein is intended to mean preventing in whole or in part, or ameliorating, reducing or controlling.

The term “about” or “approximately” means that a person skilled in the art will understand that the value is given with an acceptable error range. The range will normally be ±10% of the stated value.

The term “natural oil” as used herein means a vegetable oil extracted from seed or other parts of fruits or plants. Soybean oil, rapeseed oil and cocoa butter are examples of vegetable oils derived from seeds. Other sources are oats, palm, sunflower seed, peanut, cottonseed, palm kernel, olive, almond, avocado, cashew, corn, hazelnut, linseed, rice bran, and sesame. A specific rapeseed oil for use may be Lipex Bassol C™, which is a natural and safe clear liquid emollient intended for skin and personal care applications, composed of a carefully purified and deodorized vegetable oil. It has a high content of monosaturated fatty acids, which makes it stable against oxidation, while at the same time keeping the product as a clear liquid. It is also an emollient and it is biodegradable and non-bioaccumulating.

The term “natural ingredient” refers to ingredients present in a skin-care composition of the invention, which ingredients are found in nature, derived from natural products or they are endogenous to human. In the present context the following ingredients are regarded as natural ingredients: hydrogen peroxide, lactic acid, natural oils, beta-glucan, processed cereals, beta-glucan composition, water. Other ingredients may be diols, extracts and solvents.

The term “emollient” as used herein means a moisturizer intended for protecting, moisturizing and lubricating the skin. Emollients include white soft paraffin, castor oil, cetyl alcohol, cetostearyl alcohol, cocoa butter, isopropyl myristate, isopropyl palmitate, lanolin, liquid paraffin, polyethylene glycol, shea butter, silicone oils, stearic acid, methyl-propanediol and stearyl alcohol.

Emulsification is defined as a process of dispersing one liquid in a second immiscible liquid.

### DETAILED DESCRIPTION

The present invention provides a skin-care composition with anti-microbial effect. The composition is very skin-friendly and the majority of the ingredients in the skin-care composition are ingredients found in nature. It has especially been found that the polysaccharide beta-glucan has excellent properties regarding smoothening of the skin as well as having an emulsifying effect. Moreover, beta-glucan has no irritative effect on the skin.

In order to achieve an anti-microbial effect of the skin-care composition, the composition contains hydrogen peroxide and/or lactic acid. Hydrogen peroxide and lactic acid are well-known anti-microbial agents, but in the present set-up the hydrogen peroxide and/or lactic acid are contained in a composition which is balanced regarding the aim of achieving disinfection of the skin and at the same time maintaining or improving the skin so that irritation, inflammation, redness and drying out of the skin are avoided or at least reduced compared with other skin disinfectants. It is well known that e.g. disinfection of the skin with ethanol-based products lead to drying out of the skin. When the skin is dry, a number of other skin problems may arise such as red skin, itching of the skin and damage of the outer surface of the skin and of its barrier function, which in turn may result in infection of the skin or it may result in that it is easier for the skin to host unwanted microorganisms.

Accordingly, in the present context it is important to provide a skin-care composition, which not only can combat microorganisms, but also can preserve the skin intact, prevent skin damages and which is without any negative effects.

It has been found that it is possible to prepare a skin-care composition with excellent properties. In embodiments, the skin-care composition is based on a content of processed cereals, which surprisingly have been found to function both as a thickener, an emulsifying agent and an agent that imparts emollient effect, smoothening effect, and provides hydrating properties to the skin without any adverse effects like e.g. drying out of the skin or irritating the skin.

The present invention relates to a skin-care composition comprising beta-glucan, a natural oil, hydrogen peroxide and/or lactic acid. In one embodiment of the present invention, the skin-care composition comprises beta-glucan, lactic acid, peroxide and a natural oil. In one embodiment of the present invention, the skin-care composition comprises beta-glucan, lactic acid and a natural oil. In one embodiment of the present invention, the skin-care composition comprises beta-glucan, peroxide and a natural oil.

The skin-care composition may be in any suitable form for application to the skin. The composition may thus be in the form of a cream, an ointment, a lotion, a gel, a foam, a hydrogel, an aerosol, a spray etc. The composition may be formulated as a suspension, an emulsion, a dispersion.

The skin-care composition contains a natural oil. Even if the natural oil may be present in low concentration, the skin-care composition still has suitable properties regarding softening of the skin, smoothening of the skin, hydration of the skin etc. The composition typically is in the form of a cream, an ointment, a lotion, a foam, an aerosol or a spray and it may be formulated as a suspension, an emulsion, a dispersion or a foam. In a presently preferred form, the composition is a cream. It is contemplated that even if the concentration of oil is low, an emulsion may be formed due to the emulsifying and stabilizing effect of the beta glucan. Emulsions are very skin friendly, especially since it is an oil-in-water emulsion, where water is the continuous phase and oil is the disperse phase. In such a case, the oil is distributed in the continuous phase as small droplets, which means that the emulsion when applied to the skin is easy to apply and it easily penetrates the skin surface, i.e. the composition contains oil, but it does not appear greasy on the skin.

Establishing a stable emulsion, i.e. an emulsion which does not separate into the two phases upon storage, normally requires addition of an emulsifying agent or a surface-active agent, which “links” the oil and the water phase together. However, in a skin-care composition of the invention beta-glucan and/or processed cereals function as emulsifying agents or surface-active agents and normally, there is no need for further addition of a surface-active agent.

In a skin-care composition of the invention the concentration of the natural oil is in a range of from 3 to 15% w/w such as from 3 to 10% w/w or from 3 to 7% w/w based on the weight of the composition. Even if the oil content is relatively low, a composition of the invention has excellent properties and contributes to softening of the skin and to maintaining an intact barrier function of the skin. In embodiments, the natural oil is present in a concentration of less than 5% w/w, such as of between 2-4.9%, 1-3%, 4%, 3%, 2% or 1%.

The natural oil is typically selected from the group consisting of oat oil, rape seed oil, sunflower seed oil, palm oil, coconut oil, olive oil, shea butter oil, jojoba oil, almond oil, grapeseed oil, rose hip seed oil, argan oil, marula oil, sesame seed oil, lavender oil, tea tree oil, bergamot oil, rose oil, and avocado oil.

In embodiments, the skin-care composition contains a natural oil selected from rape seed oil or sunflower seed oil.

Beta-glucan is typically derived from cereal grains. The cereal grains may be wheat, oat, rye or barley.

Preferably, beta-glucan is derived from oats. Typically, the concentration of beta-glucan in a skin-care composition of the invention is from about 0.3 to 20% w/w such as from 0.5 to 15% w/w, from 0.5 to 10% w/w or from 0.75 to 5% w/w based on the weight of the total composition.

Beta-glucan may be used in purified form or it may be used in the form of a composition containing processed cereal grains. Beta-glucan may also in itself be processed cereal grains. WO 2016/124608 discloses methods for obtaining beta-glucan from cereals, notably from oats. When beta-glucans are processed from cereal grains, they may be de-hulled and parts of the proteins may have been removed.

The cereal grains are processed in accordance with WO 2016/124608 to remove the hull of the grains and to remove parts of the protein and de-gradated starch.

Such a beta-glucan containing composition may contain beta-glucan admixed with other ingredients or beta-glucan may be a part of the processed cereal grains. Such beta-glucan compositions are in the form of a powder. The beta-glucan and/or the composition containing the beta-glucan seems to impart emulsifying properties to the skin-care composition, when a natural oil is present. Thus, it is very easy to disperse the beta-glucan or the beta-glucan composition in the natural oil and when water is added the mixture a homogeneous mixture is obtained, i.e. no separation in an oil phase and a water phase. Therefore, it is contemplated that an emulsion is formed even in those cases where only a small concentration of oil is present. Moreover, the content of hydrogen peroxide and lactic acid do not change the ability of forming an emulsion. The emulsion also seems to have a suitable stability—both relating to the emulsion (no separation) and relating to the content of the active ingredients (beta-glucan, hydrogen peroxide and lactic acid).

In the current context, an emulsion is a mixture of two or more liquids that are normally immiscible owing to liquid-liquid phase separation. Emulsions are part of a more general class of two-phase systems of matter called colloids. Although the terms colloid and emulsion can be used interchangeably, emulsion should be used when both phases, dispersed and continuous, are liquids. In an emulsion, one liquid (the dispersed phase) is dispersed in the other (the continuous phase). In the current context an oil-in-water emulsion is formed, in which water is the dispersed phase and oil is the continuous phase. Theoretically, multiple emulsions are also possible, including a “water-in-oil-in-water” emulsion and an “oil-in-water-in-oil” emulsion.

The oil droplets dispersed in the continuous phase (sometimes referred to as the “dispersion medium”) assumed to be statistically distributed to produce roughly spherical droplets.

In the current context, emulsion stability refers to the ability of the emulsion to resist change in its properties over time. There are four types of instability in emulsions: flocculation, coalescence, creaming/sedimentation, and Ostwald ripening. The beta-glucan increases the kinetic stability of the emulsion so that the size of the droplets does not change significantly with time. The stability of an emulsion, like a suspension, can be studied in terms of zeta potential, which indicates the repulsion between droplets or particles. If the size and dispersion of droplets does not change over time, it is said to be stable, such as but not limited to over 28 days storage at 25° C.

The concentration of beta-glucan in a beta-glucan composition is from about 20 to about 45% w/w such as from about 30 to about 40% w/w, from about 32 to about 38% w/w, from about 32 to about 36% w/w such as about 34% w/w. When a beta-glucan composition is used, the beta-glucan composition is used in a skin-care composition in a concentration from about 0.5% to about 65% w/w such as from about 10% to about 55%, from about 15% to about 50%, from about 20% to about 45% w/w, from about 30% to about 45% such as about 40% w/w.

As mentioned above, the processed cereal grains are de-hulled and parts of the proteins are removed. Thus, the concentration of protein in the beta-glucan containing composition is in a range of from 1 to 10% w/w, such as from 2 to 8% w/w, from 3 to 7% w/w such as about 2.5%. The content of protein in the skin-care composition is from 0.05% to about 6.5% w/w such as from 0.75 to about 5% w/w, from 0.75% to about 3% w/w, from 0.75% to about 2% w/w such as about 1% w/w.

The processed cereal grains furthermore contain dietary fibers. The total concentration of dietary fibers in a beta-glucan containing composition is in a range of from 20 to 60% w/w such as from 25% to 55% w/w, from 30% to 50% w/w such as about 40% w/w. The water-soluble fibers impart structure to the skin-care composition and contribute to thickening of the composition.

Moreover, the processed cereal grains contain carbohydrates. The total amount of carbohydrates in a beta-glucan containing composition is in a range of from 60 to 90% w/w.

Processed cereal grain may furthermore contain natural oils or fat. Typically, the concentration of natural oils or fat in a beta-glucan containing composition is from about 1% to about 10% w/w such as from about 2 to about 7% w/w.

A suitable beta-glucan containing composition for use according to the invention is the product PromOat® manufactured by Lantmännen. However, other compositions may be used provided the content of proteins, fibers and natural oils (fat) is within the ranges given above.

PromOat® contains processed oat grains, and the beta-glucan containing composition has the following constituents:

| Nutritional Information | Nutrients per 100 grams | |
|---|---|---|
| Calories | 323 | kcal |
| Total Fat | 6.5 | g |
| Saturated Fat | 1.2 | g |
| Trans Fat | 0 | g |
| Cholesterol | 0 | mg |
| Sodium | 68 | mg |
| Total Carbohydrate | 82.5 | g |
| Dietary Fiber | 40 | g |
| Total Sugars | 2.0 | g |
| Protein | 3.5 | g |
| Vitamin D | 0 μg | (0 IU) |
| Calcium | 9.5 | mg |
| Iron | 0 | mg |
| Potassium | 765 | mg |
| Moisture | 5.5 | g |
| Ash | 2.0 | g |
| Beta-Glucan | 34 | g |

In the amount of dietary fibers beta-glucan is included.

In order to avoid addition of non-natural products to the skin-care composition of the invention it is preferred to use beta-glucan in a beta-glucan containing composition as discussed above. By doing so, only natural products already present in cereals are included in a skin-care composition of the invention (apart from hydrogen peroxide, lactic acid, and, if present, small amounts of thickeners or other acceptable excipients). It is of interest to note that it is possible to avoid use of e.g. preservatives and non-natural surface-active agents etc., but solely rely on ingredients naturally occurring in nature even if a relatively sophisticated formulation is obtained in emulsion form.

This being said, a skin-care composition of the invention may also be made with the use of purified beta-glucan, i.e. where beta-glucan is not contained in a beta-glucan containing composition. In such cases, it may be necessary to add further ingredients to a skin-care composition of the invention such as e.g. a thickener.

A thickener may be derived from processed cereals (as described above), or it may be selected from biopolymers including polysaccharides of microbial source; vegetable gums, starches, pectins, agars or gelatins. A vegetable gum is typically selected from alginate, guar gum locust bean gum, diutan gum, welan gum and xanthan gum.

Other ingredients may be included as well, if needed. They may be selected from solvents, surface-active agents, preservatives, emollients such as glycerol or propylene glycol, hydrating agents, fragrance etc.

A skin-care composition of the present invention also contains antimicrobial agents. Hydrogen peroxide is a chemical compound with the formula H₂O₂. In its pure form it is a pale blue liquid. It is slightly more viscous than water. Due to the presence of peroxide bonds, hydrogen peroxide is by nature unstable and decomposes slowly in the presence of light. Hydrogen peroxide is typically stored with a stabilizer in a weakly acidic solution in a dark coloured bottle to avoid substantial degradation. Hydrogen peroxide decomposes to water and oxygen.

Hydrogen peroxide has strong oxidizing properties and is used for bleaching e.g. in laundry detergents. It is also used as a disinfectant and may be used for the sterilization of various surfaces, including surgical tools, and may be deployed as a vapor for room sterilization. Hydrogen peroxide demonstrated broad-spectrum efficacy against viruses, bacteria, yeasts, and bacterial spores. In general, greater activity is seen against Gram-positive than Gram-negative bacteria; however, the presence of catalase or other peroxidases in these organisms may increase tolerance in the presence of lower concentrations. Lower levels of concentration (3%) will work against most spores; higher concentrations (7 to 30%) and longer contact times will improve sporicidal activity.

it is generally recognized as a safe antimicrobial agent by the U.S. Food and Drug Administration (FDA).

When hydrogen peroxide is used in a composition care must be taken to avoid unnecessary degradation of hydrogen peroxide. However, hydrogen peroxide seems to be stable in a skin-composition of the present invention. In the present context the term “stable” refers to that the original content of hydrogen peroxide may vary ±10% during the storage period at room temperature. Preferably, the skin-composition is contained in a container which is dark or the like to exclude the content of the container from being exposed to sun light. Moreover, pH of the skin-care composition of the invention is kept at the acidic range such as a pH in a range from 3 to 5 such as about 3, 3.5, 4, 4.5, or 5. It should be noted that even if pH is acidic, the skin-care composition of the invention is skin-friendly and does not impart any redness to the skin.

Hydrogen peroxide is present in a skin-care composition of the invention in a concentration of from 0.5% to 25% w/w such as from 0.5% to 20% w/w. from 0.5% to 15% w/w such as from 1% to 10% w/w, from 1% to 8% w/w, from 1.5% to 6% w/w, from 2% to 3.5% w/w or from 2.5% to 3% w/w.

Normally, it is a challenge to formulate stable compositions with hydrogen peroxide. Hydrogen peroxide seems to degrade polysaccharides and, in a skin-care composition of the invention polysaccharides are present at least in the form of beta-glucan. The polysaccharides in a skin-care composition of the invention contributes both to the advantageous properties when the skin-care composition is applied on the skin (e.g. smoothening effect, softening effect etc.) and to obtaining a suitable texture of the skin-friendly composition e.g. regarding viscosity.

The viscosity of a skin-care composition of the invention depends on whether it is presented as a solution, an emulsion, a suspension, a cream, a lotion, a spray etc. Typically, solutions, lotions and sprays have lower viscosities than emulsions, suspensions and creams.

Besides the antimicrobial agent hydrogen peroxide or on its own, lactic acid is in embodiments of the invention present in a skin-care composition of the invention. Dependent of the pH of the final composition, lactic acid may be present as lactate, lactic acid and/or a mixture thereof; its pK_{A} value is 3.86. Lactic acid is an organic acid. Lactic acid is chiral, consisting of two enantiomers. One is known as L-(+)-lactic acid or (S)-lactic acid and the other is D-(−)-lactic acid or (R)-lactic acid. A mixture of the two in equal amounts is called DL-lactic acid or racemic lactic acid. In humans L-lactate is constantly produced from pyruvate via the enzyme lactate dehydrogenase during normal metabolism and exercise. During power exercise when the rate of demand for energy is high, glucose is broken down and oxidized to pyruvate and lactate is then produced from the pyruvate faster than the body can process it, causing the lactate levels to rise. Lactate can also serve as an important source of energy for other organs, including the heart and liver. During physical activity up to 60% of the heart muscle's energy turnover rate derives from lactate oxidation.

Lactic acid has been shown to be effective as an antimicrobial agent against both gram-negative and gram-positive bacteria as well as it has anti-viral effect. Moreover, it is believed that lactate has a positive effect on the skin and contributes to keep the skin healthy and smooth. It also has moisturizing effect on the skin.

In embodiments of the invention, a skin-care composition of the invention contains a lactic acid. When a salt of lactic acid is used it may be in the form of an alkali metal salt such as sodium lactate or potassium lactate or it may be an alkaline earth metal salt such as magnesium lactate or calcium lactate. Sodium lactate is preferred. The concentration of lactate (calculated as sodium lactate) in a skin-care composition of the invention is in a range of from 0.1% to 5% w/w such as from 0.1 to 3% w/w, from 0.1 to 2% w/w, from 0.2 to 1% w/w or from 0.1% to 0.5% w/w.

Lactic acid is a fully biodegradable molecule, it is non-toxic and approved as a food ingredient and has GRAS status. L(+) lactic acid is approved for e.g. food and feed area disinfectants, It antibacterial action depends inter alia on pH of the solution,

A skin-care composition of the invention typically has a pH value in a range of from 2.5 to 5.5 such as from 2.5, 2.6, or 2.7 to 3.0, 3.5, 4.0. 4.5, or 5, or from 3.0 to 4.0. A skin-care composition of the invention in some embodiments has a pH value of 5.0, 5.5, 6.0 or 6.5.

As mentioned herein before, a skin-care composition of the invention may also contain other ingredients such as e.g., solvents, preservatives, stabilizing agents, fragrances, pH adjusting agents, foam-formers, thickeners, surface-active agents etc.

A skin-care composition according to the invention may contain further antimicrobial agents. In embodiment a composition may contain levullinic acid, with has both antibacterial and antifungal properties as well as it is used in cosmetics as a fragrance.

A skin-care composition of the invention may comprise a disinfectant selected from ethanol, isopropyl alcohol, chlorine including chlorohexidine. In one embodiment, the composition comprises no more than 10%, such as no more than 1, 2, 3, 4, 5, 6, 7, 8, 9 or 10%, such as 35 ethanol.

However, the main approach is to provide a skin-care composition wherein the majority of the ingredients are naturally based. Therefore, the content of artificial ingredients should be kept to a minimum.

Thus, the content of hydrogen peroxide, lactic acid, beta-glucan, processed cereals or other natural material functioning as a thickener, and/or a surface-active agent, and/or a moisturizing agent or the like, natural oil and solvent such as water should preferably be at least 80% w/w of the total weight of the composition. In embodiments these ingredients constitute at least 85% w/w such as at least 90% w/w, at least 92% w/w. at least 93% w/w, at least 94% w/w, at least 95% w/w, at least 96% w/w, at least 97% w/w, at least 98% w/w or at least 99% w/w of the total weight of the skin-care composition.

A skin-care composition of the invention may also contain a thickener such as a biopolymer including polysaccharides of microbial source; vegetable gums such as e.g. alginate, guar gum, locust bean gum, dilutan gum, welan gum or xanthan gum may be present and/or a surface-active agent like e.g. a fatty acid may be present and/or a moisturizing agent like a natural oil. In some cases, when beta-glucan is used in the form of processed cereal grains the content of water-soluble fibers may be sufficient to obtain a suitable viscosity of the skin-care composition. A suitable solvent is an aqueous solvent such as water. Typically, water is present in a skin-care composition of the invention in a concentration of from 45-95% w/w.

A skin-care composition according to the invention is typically used as a sanitizer. Typically, it is used as a hand sanitizer, but it may be used to the skin in general.

As shown in the Examples herein, the skin-care composition is active against bacteria and due to its content of hydrogen peroxide and lactate/lactic it is contemplated to be active against viruses as well, i.e. functioning as an anti-microbial composition. It has been shown that a synergistic effect between hydrogen peroxide and lactic acid in bacteria such as E. coli, P. aeruginosa, E. hirae, S. aureus, L. monocytogenes and S. typhimurium, see the following tables:

TABLE 1

| Log reduction | | | | |
|---|---|---|---|---|
| according to EN 1276 | E. coli | P. aeruginosa | E. hirae | S. aureus |
| 3% LA + 3% H₂O₂ | >5 | >5 | >5 | >5 |
| 3% LA + 3% H₂O₂, | >5 | >5 | >5 | >5 |
| pH 3 | | | | |
| 3% H₂O₂ | >5 | >5 | 0.1 | <1 |

TABLE 2

| Log reduction | | |
|---|---|---|
| according to EN 1276 | L. monocytogenes | S. typhimurium |
| 1% LA | 3.8 | >5 |
| 1% LA, pH 3 | 2.2 | >5 |
| 1% LA + 1% H₂O₂ | >5 | >5 |
| 1% LA + 1% H₂O₂, pH 3 | >5 | >5 |
| 1% H₂O₂ | 0.6 | 0.5 |

The composition is typically delivered via a suitable device such as a bottle equipped with a pump, a tube, a spray bottle or the like. The composition is applied to the skin directly from the device and rubbed into the skin. The dosage depends on the area of the skin but is typically sufficient to cover the area. For hand disinfection it is contemplated that 1 ml of the composition is sufficient per disinfection.

Specific compositions appear from the Examples herein which are intended to illustrate the invention and not construed to limit the invention in any way.

### EXAMPLES

### Example 1—Skin-Care Composition—I

A composition was prepared from the following ingredients:
i) from 0.3 to 20% w/w of beta-glucan,
ii) from 3 to 15% w/w of a natural oil,
iii) from 0.1 to 5% w/w lactate/lactic acid (calculated as sodium lactate),
iv) from 45 to 95% w/w of water,
v) from 0.5 to 25% w/w of hydrogen peroxide
vi) optionally, from 0.05 to 0.50% w/w of a thickener, and
vi) optionally from 0.1-5% w/w of a fragrance,
wherein the % w/w is based on the total weight of the composition.

Beta-glucan was dispersed in rape seed oil where after water was added under stirring followed by addition of the other ingredient. The resulting composition was a white—yellowish cream. Compositions were also made with a content of the thickener (Composition Ia), with a content of the fragrance (Composition Ib) and with a content of both the thickener and the fragrance (Composition Ic). In composition Id beta-glucan was employed in the form of processed oat grain, PromOat®, and the concentration of beta-glucan-containing composition (i.e. PromOat®) in the skin-care composition was in a range of from 20 to 60% w/w.

In Example 1, a thickener may be included notably guar gum, diutan gum, welan gum or xanthan gum.

The skin-care composition described in Example 1 was made but using a beta-glucan composition as a source of beta-glucan. The beta-glucan composition typically contains 32-36% w/w beta-glucan and de-hulled oat from which parts of proteins and degraded starch have been removed.

A suitable beta-glucan composition is PromOat®. PromOat® is a product manufactured by Lantmännen Oats, Sweden. PromOat® is an oat-based product containing de-hulled oat and wherein parts of the proteins and degraded starch have been removed. It contains 32-36% w/w beta-glucan and has the following nutritional information:

TABLE 3

| Nutritional Information | Nutrients per 100 grams | |
|---|---|---|
| Calories | 323 | kcal |
| Total Fat | 6.5 | g |
| Saturated Fat | 1.2 | g |
| Trans Fat | 0 | g |
| Cholesterol | 0 | mg |
| Sodium | 68 | mg |
| Total Carbohydrate | 82.5 | g |
| Dietary Fiber | 40 | g |
| Total Sugars | 2.0 | g |
| Protein | 3.5 | g |
| Vitamin D | 0 μg | (0 IU) |
| Calcium | 9.5 | mg |
| Iron | 0 | mg |
| Potassium | 765 | mg |
| Moisture | 5.5 | g |
| Ash | 2.0 | g |
| Beta-Glucan | 34 | g |

In the table 3, above, the content of beta-glucan is included in the content of dietary fiber.

### Example 2—Skin-Care Compositions

The skin-care composition of Example 1 was made but using Persynt® 350 COS from Evonik as a source of hydrogen peroxide. It contains 50% or hydrogen peroxide in water and fulfils the requirement of German Pharmacopoeia and European Pharmacopoeia.

### Example 3—Bactericidal Activity

A skin-care composition with the following composition was tested for bactericidal activity:
i) 4% w/w PromOat® containing 32-36% beta-glucan,
ii) 10% w/w rape seed oil,
iii) 0.25% w/w lactate (calculated as sodium lactate),
iv) 77.5% w/w water,
v) 0.1% w/w xanthan gum,
vi) 8% w/w Persynt 350 COS leading to a concentration of 2.80% w/w hydrogen peroxide,
wherein the % w/w is based on the total weight of the composition. pH of the skin-care composition was 4.0.

The composition was tested under the following experimental conditions:
Diluent used for product test solutions: distilled water
Product test concentrations: 10%, 50% and 80% (v/v)
Test organisms: Staphylococcus aureus ATCC 6538
   Pseudomonas aeruginosa ATCC 15442
Temperature of incubation: 37° C.±1° C.
Test conditions:
Test temperature: 20° C.±1° C.
Contact time: 5 min±10 s

Special Remarks:

All tests and validations were between the basic limits.

The product shows at least at one concentration a reduction less than 5 log.

There was no precipitation during the test.

Test for Staphylococcus aureus ATCC 6538

Test suspension and test (N and N₀)

TABLE 4

| N | Vc1 | Vc2 |
|---|---|---|
| 10⁻⁶ | >330 | >330 |
| 10⁻⁷ | 42 | 40 |

X_{wm}=4.10E+08

log N=8.61

N₀=N/10

log N₀=7.61

TABLE 5

| Product | | | | | | |
|---|---|---|---|---|---|---|
| concentration | | | Na = | | | |
| (%) | Vc1 | Vc2 | X × 10 | logNa | logR | Contact time |
| 10 | >330 | >330 | >3300 | >3.52 | <4.09 | 5 min ± 10 s |
| 50 | >330 | >330 | >3300 | >3.52 | <4.09 | 5 min ± 10 s |
| 80 | >330 | >330 | >3300 | >3.52 | <4.09 | 5 min ± 10 s |

Test for Pseudomonas aeruginosa ATCC 15442

Test suspension and test (N and No)

TABLE 6

| N | Vc1 | Vc2 |
|---|---|---|
| 10⁻⁶ | 296 | 320 |
| 10⁻⁷ | 32 | 39 |

X_{wm}=3.12E+08

log N=8.49

N₀=N/10

log N₀=7.49

TABLE 7
Test results:

| Product | | | | | | |
|---|---|---|---|---|---|---|
| concentration | | | Na = | | | |
| (%) | Vc1 | Vc2 | X × 10 | logNa | logR | Contact time |
| 10 | >330 | >330 | >3300 | >3.52 | <3.97 | 5 min ± 10 s |
| 50 | >330 | >330 | >3300 | >3.52 | <3.97 | 5 min ± 10 s |
| 80 | 42 | 28 | 350 | 2.54 | 4.95 | 5 min ± 10 s |

### Explanations

Vc=counting per ml (one plate or more)
X=mean of Vc1 and Vc2 (1st and 2 nd double determinations)
X_{wm}=weighted mean of Vc1 and Vc2
log R=logarithmic reduction (log R=log N₀−log Na)

### Example 4—Test for Irritation

The composition of Example 2 was tested for skin irritation in 8 healthy volunteers.

The composition was applied after hand wash in a time period of 7 days and the effect scored 1-5, where 1 is poor and 5 is very good.

The results were as follows:

TABLE 8
Result

| | |
|---|---|
| How well do you experience the cream | 4.1 |
| Is the cream easy to apply | 4.7 |
| Does the cream smoothen the skin | 4.5 |
| Does the cream give any skin irritation | 100% responded NO |
| Does the cream make the skin red | 100% responded NO |
| Would you recommend the cream to others | 70% responded YES |

### Example 5—Skin Care Composition of the Invention

A skin-care composition with the following composition was tested for bactericidal activity:
i) 4% w/w PromOat® containing 32-36% beta-glucan,
ii) 10% w/w rape seed oil, Lipex Bassol C from AAK
iii) 0.5% w/w lactate (calculated as sodium lactate),
iv) 1.5% w/w lactic acid, 80% Purac
iv) 75% w/w water,
v) 1% w/w diutan gum, Kelco care Diutan gum
vi) 8% w/w Persynt 350 COS leading to a concentration of 2.8-3.0% w/w hydrogen peroxide,
wherein the % w/w is based on the total weight of the composition. pH of the skin-care composition was 3.1.

The composition was made as described in Examples 1˜4 herein.

The viscosity of the composition was measured with a Brookfield viscometer using different spindles. A viscosity of 30,000 mPa was found at a temperature of 20-22° C.

### Example 6—Skin Care Composition of the Invention

A skin-care composition with the following composition was made:
i) 4% w/w PromOat® containing 32-36% beta-glucan,
ii) 10% w/w rape seed oil, Lipex Bassol C from AAK
iii) 0.5% w/w lactate (calculated as sodium lactate),
iv) 2.0% w/w lactic acid, 80% Purac
iv) 75% w/w water,
v) 1% w/w diutan gum, Kelco care Diutan gum
vi) 8% w/w Persynt 350 COS leading to a concentration of 2.8-3.0% w/w hydrogen peroxide,
wherein the % w/w is based on the total weight of the composition. pH of the skin-care composition was 3.1.

### Example 7—Bactericidal Activity

The skin-care composition of Example 5 was tested for bactericidal activity. The composition was tested under the following experimental conditions:
Diluent used for product test solutions: distilled water
Product test concentrations: 10%, 50% and 80% (v/v)
Test organisms: Staphylococcus aureus ATCC 6538
   Pseudomonas aeruginosa ATCC 15442
Temperature of incubation: 37° C.±1° C.
Test conditions:
Test temperature: 20° C.±1° C.
Contact time: 5 min±10 s

Special Remarks:

All tests and validations were between the basic limits.

The product shows at least at one concentration a reduction less than 5 log.

There was no precipitation during the test.

Test for Staphylococcus aureus ATCC 6538

Test suspension and test (N and No)

TABLE 9

| N | Vc1 | Vc2 |
|---|---|---|
| 10⁻⁶ | 256 | 224 |
| 10⁻⁷ | 30 | 25 |

X_{wm}=2.43E+08

log N=8.39

N₀=N/10

log N₀=7.39

TABLE 10

| Product | | | | | | |
|---|---|---|---|---|---|---|
| concentration | | | Na = | | | |
| (%) | Vc1 | Vc2 | X × 10 | logNa | logR | Contact time |
| 10 | >330 | >330 | >3300 | >3.52 | <3-87 | 5 min ± 10 s |
| 50 | >330 | >330 | >3300 | >3.52 | <3.87 | 5 min ± 10 s |
| 80 | 312 | 328 | 3200 | 3.51 | 3.88 | 5 min ± 10 s |

Test for Pseudomonas aeruginosa ATCC 15442

Test suspension and test (N and No)

TABLE 11

| N | Vc1 | Vc2 |
|---|---|---|
| 10⁻⁶ | 274 | 286 |
| 10⁻⁷ | 31 | 35 |

X_{wm}=2.85E+08

log N=8.45

N₀=N/10

log N₀=7.45

TABLE 12
Test results:

| Product | | | | | | |
|---|---|---|---|---|---|---|
| concentration | | | Na = | | | |
| (%) | Vc1 | Vc2 | X × 10 | logNa | logR | Contact time |
| 10 | 45 | 49 | 470 | 2.67 | 4.78 | 5 min ± 10 s |
| 50 | 0 | 0 | >140 | >2.15 | >5.30 | 5 min ± 10 s |
| 80 | 0 | 0 | >140 | >2.15 | >5.30 | 5 min ± 10 s |

### Explanations

Vc=counting per ml (one plate or more)
X=mean of Vc1 and Vc2 (1^{st} and 2^{nd} double determinations)
X_{wm}=weighted mean of Vc1 and Vc2
log R=logarithmic reduction (log R=log N₀−log Na)

### Example 8

Sprayable compositions were made using the recipes described in examples 1-6, but the content of thickener (gum) was excluded. The resulting compositions can be administered via a spray device.

1. A skin-care composition comprising beta-glucan, a natural oil and hydrogen peroxide and/or lactic acid, wherein the concentration of the natural oil is in a range from 3 to 15% w/w based on the weight of the composition and wherein the concentration of the beta-glucan is from 0.3 to 20% w/w.

2. A skin-care composition according to claim 1, comprising beta-glucan, a natural oil, hydrogen peroxide and lactic acid.

3. A skin-care composition according to claim 1, wherein the concentration of lactic acid is from 0.1 to 5% w/w.

4. A skin-care composition according to claim 1, wherein the concentration of hydrogen peroxide is from 0.5 to 15% w/w.

5. A skin-care composition according to claim 1, in the form of an oil-in-water emulsion.

6. A skin-care composition according to claim 1, wherein the natural oil is selected from oat oil, rape seed oil, sunflower seed oil, palm oil, coconut oil, olive oil, rhea butter oil, jojoba oil, almond oil, grapeseed oil, rose hip seed oil, argan oil, marula oil, sesame seed oil, lavender oil, tea tree oil, bergamot oil, rose oil, or avocado oil.

7. A skin-care composition according to claim 6, wherein the natural oil is rape seed oil or sunflower seed oil.

8. A skin-care composition according to claim 1, comprising one or more processed cereals.

9. A skin-care composition according to claim 8, wherein the cereal is selected from oat, barley, wheat, or rye, or mixtures thereof.

10. A skin-care composition according to claim 9, wherein the cereal is oat.

11. A skin-care composition according to claim 9, where grains of the cereal are processed to remove hull, parts of the protein and degraded starch.

12. A skin-care composition according to claim 8, wherein the one or more processed cereals contains from 1 to 10% w/w proteins.

13. A skin-care composition according to claim 8, wherein the one or more processed cereals contain the following: fat, carbohydrate, fibers, sugar, and protein.

14. A skin-care composition according to claim 8, wherein beta-glucan is contained in or admixed with one or more processed cereals.

15. A skin-care composition according to claim 14, wherein beta-glucan is contained in a composition containing processed cereal and the concentration of beta-glucan in the composition is from 32 to 36% w/w.

16. A skin-care composition according to claim 8, wherein the cereal is oat.

17. A skin-care composition according to claim 1, wherein beta-glucan is contained in a composition containing a processed cereal, and wherein the composition has the following constituents:

| Nutritional Information | Nutrients per 100 grams | |
|---|---|---|
| Calories | 323 | kcal |
| Total Fat | 6.5 | g |
| Saturated Fat | 1.2 | g |
| Trans Fat | 0 | g |
| Cholesterol | 0 | mg |
| Sodium | 68 | mg |
| Total Carbohydrate | 82.5 | g |
| Dietary Fiber | 40 | g |
| Total Sugars | 2.0 | g |
| Protein | 3.5 | g |
| Vitamin D | 0 μg | (0 IU) |
| Calcium | 9.5 | mg |
| Iron | 0 | mg |
| Potassium | 765 | mg |
| Moisture | 5.5 | g |
| Ash | 2.0 | g |
| Beta-Glucan | 34 | g |

18. A skin-care composition according to claim 1 further comprising a thickener selected from biopolymers including polysaccharides of microbial source; vegetable gums, starches, pectins, agars or gelatins.

19. A skin-care composition according to claim 18, wherein the thickener is selected from alginate, guar gum locust bean gum, diutan gum, welan gum and xanthan gum.

20. A skin-care composition according to claim 1 further comprising a disinfectant.

21. A skin-care composition according to claim 20, wherein the disinfectant is selected from ethanol, isopropyl alcohol, chlorine, and chlorohexidine.

22. A skin-care composition according to claim 1 further comprising a fragrance.

23. A skin-care composition according to claim 22, wherein the fragrance has antibacterial and/or fungicide effect.

24. A skin-care composition according to claim 22, wherein the fragrance is levulinic acid.

25. A skin-care composition according to claim 1 in the form of a lotion, a cream or a hydrogel.

26. A skin-care composition according to claim 25 in the form of a cream.

27. A skin-care composition according to claim 1 containing
i) from 0.3 to 20% w/w of beta-glucan,
ii) from 3 to 15% w/w of a natural oil,
iii) from 1 to 5% w/w lactic acid,
iv) from 45 to 95% w/w of water,
v) from 0.5 to 15% of hydrogen peroxide, and
vi) from 0-5% w/w of a fragrance,
wherein the % w/w is based on the total weight of the composition.

28. A skin-care composition according to claim 1 containing
i) from 0.5 to 15% w/w beta-glucan,
ii) from 5 to 15% w/w of a natural oil,
iii) from 1 to 5% w/w lactic acid
iv) from 0.05% to 0.50% w/w of a thickener,
v) from 2 to 5% w/w hydrogen peroxide, and
vi) from 70 to 85% w/w water,
wherein the % w/w is based on the total weight of the composition.

29. A skin-care composition according to claim 1, wherein the natural oil is rape seed oil.

30. A skin-care composition according to claim 1, having a pH in a range of from 2 to 6.

31. A method of disinfection of skin comprising administering a skin-care composition according to claim 1 to a patient in need of such treatment.

32. (canceled)

33. A skin-care composition according to claim 3, wherein the concentration of lactic acid is from 0.25 to 5% w/w.

34. A skin-care composition according to claim 3, wherein the concentration of lactic acid is from 1 to 5% w/w.
